(19) United States
(12) Patent Application Publication
(10) Pub. No.: US 2021/0372420 A1
(43) Pub. Date: Dec. 2, 2021

(54) OIL MIST MEASUREMENT DEVICE AND METHOD AND COMPRESSION SYSTEM

(71) Applicant: KABUSHIKI KAISHA KOBE SEIKO SHO (KOBE STEEL, LTD.), Hyogo (JP)

(72) Inventors: Kaname ARAKI, Kobe-shi (JP); Kazuhei TAKAHASHI, Takasago-shi (JP); Yuya NAKAMOTO, Takasago-shi (JP)

(73) Assignee: KABUSHIKI KAISHA KOBE SEIKO SHO (KOBE STEEL, LTD.), Hyogo (JP)

(21) Appl. No.: 17/216,776

(22) Filed: Mar. 30, 2021

(30) Foreign Application Priority Data
May 29, 2020 (JP) 2020-093983

(51) Int. Cl. F04D 29/063; F04D 29/58; F04D 27/00

(52) U.S. Cl. CPC F04D 29/063; F04D 27/001; F04D 29/5886

(57) ABSTRACT

Provided are oil mist measurement device and method capable of obtaining an oil mist concentration with higher accuracy and a compression system using the oil mist measurement device.

The oil mist measurement device of the present invention includes first-A and first-B detection sections 1A, 1B configured to detect measurement target pressures, a second detection section 2 configured to irradiate a measurement target with detection light to detect the intensity of light scattered by oil mist in the measurement target, and a concentration processing section 32 configured to correct the scattered light intensity detected by the second detection section 2 based on the pressures detected by the first-A and first-B detection sections 1A, 1B to obtain a corrected scattered light intensity thereby obtaining an oil mist concentration based on the obtained corrected scattered light intensity.

### BACKGROUND OF THE INVENTION

### Field of the Invention

The present invention relates to oil mist measurement device and method for measuring oil mist in compressed gas and a compression system using the oil mist measurement device.

### Description of the Related Art

Compressors configured to compress gas (a gaseous body) can be classified according to various points of view. In terms of lubrication, the compressors can be classified into two types of a non-oil supply type and an oil supply type. The oil supply compressors inject oil into compression chambers to perform, with the oil, cooling of compression heat, internal lubrication, and sealing. As one of these compressors, an oil-cooling compressor has been generally broadly used in an industrial field due to characteristics such as a high efficiency, space saving, and long-term operation performance. In the oil supply compressor, oil is supplied into gas for the purpose of, e.g., lubrication and removal of compression heat. Thus, after compression, the oil needs to be separated and removed from the compressed gas until reaching an acceptable oil content. For determining whether or not the oil has been removed until reaching the acceptable oil content, oil mist in the compressed gas needs to be measured. As one technique, JP 2016-153774 A discloses, for example, an oil mist detection device configured to detect the light scattered by oil mist to obtain an oil mist concentration.

The oil mist concentration is measured in units of ppmwt, and is defined as an oil mist weight flow rate with respect to a compressed gas weight flow rate per unit time. When a compressed gas pressure fluctuates, the compressed gas weight flow rate changes due to a change in a compressed gas density. However, the oil mist weight flow rate is measured from the light scattered on oil mist particles, and for this reason, does not change as in the change in the compressed gas weight flow rate. Thus, there is a probability that the oil mist concentration is not obtained with favorable accuracy.

The present invention has been made in view of the above-described situation, and is intended to provide oil mist measurement device and method capable of obtaining an oil mist concentration with higher accuracy and a compression system using the oil mist measurement device.

As a result of various types of study, the inventor(s) of the present invention has found that the above-described object is achieved by the following aspects of the present invention. That is, an oil mist measurement device according to one aspect of the present invention includes a first detection section configured to detect a measurement target pressure, a second detection section configured to irradiate a measurement target with detection light to detect the intensity of light scattered by oil mist in the measurement target, and a concentration processing section configured to correct the scattered light intensity detected by the second detection section based on the pressure detected by the first detection section to obtain a corrected scattered light intensity, thereby obtaining an oil mist concentration based on the obtained corrected scattered light intensity

Such an oil mist measurement device corrects the intensity of the light scattered by the oil mist in the measurement target based on the measurement target pressure, thereby obtaining the oil mist concentration. Thus, the oil mist concentration can be obtained with higher accuracy.

In another aspect, in the above-described oil mist measurement device, the concentration processing section performs the correction by using a correspondence between a change in the measurement target pressure and a change in the output of the second detection section.

Such an oil mist measurement device obtains the output of the second detection section at a predetermined reference pressure set as a reference so that the output detected by the second detection section can be converted into the output at the reference pressure and the correction can be made by means of the pressure detected by the first detection section and the correspondence.

An oil mist measurement method according to another aspect of the present invention includes the first detection step of detecting a measurement target pressure, the second detection step of irradiating a measurement target with detection light to detect the intensity of light scattered by oil mist in the measurement target, and the concentration processing step of correcting the scattered light intensity detected at the second detection step based on the pressure detected at the first detection step to obtain a corrected scattered light intensity thereby obtaining an oil mist concentration based on the obtained corrected scattered light intensity.

Such an oil mist measurement method corrects the intensity of the light scattered by the oil mist in the measurement target based on the measurement target pressure, thereby obtaining the oil mist concentration. Thus, the oil mist concentration can be obtained with higher accuracy.

A compression system according to another aspect of the present invention includes an oil supply compressor and any of the above-described oil mist measurement devices. The measurement target is gas compressed in the oil supply compressor. Preferably, in the above-described compression system, the oil supply compressor is an oil-cooling gas compressor. Preferably, in the above-described compression system, the oil supply compressor is an oil-cooling screw compressor.

According to such an aspect, the compression system including any of the above-described oil mist measurement devices can be provided.

In another aspect, the above-described compression system further includes an abnormality processing section configured to obtain a cumulative oil mist amount accumulated over predetermined time based on the oil mist concentration detected by the oil mist measurement device, thereby performing predetermined abnormality processing in a case where the obtained cumulative oil mist amount exceeds a predetermined threshold. Preferably, in the above-described compression system, the abnormality processing is the processing for decreasing the rotational speed of the oil supply compressor. Preferably, in the above-described compression system, the abnormality processing is the processing for stopping the oil supply compressor. Preferably, the above-described compression system further includes an output section configured to make predetermined output, and the abnormality processing is the processing fir informing an abnormality regarding the oil mist concentration from the output section.

Such a compression system performs the predetermined abnormality processing in a case where the cumulative oil mist amount exceeds the predetermined threshold, and therefore, can handle the oil mist abnormality.

The oil mist measurement device and method according to the present invention can obtain the oil mist concentration with higher accuracy According to the present invention, the compression system using the oil mist measurement device can be provided.

### BRIEF DESCRIPTION OF THE DRAWINGS

FIG. 1 is a block diagram illustrating a configuration of a compression system in an embodiment.

FIG. 2 is a graph for describing a correction method in calculation of an oil mist concentration.

FIG. 3 is a flowchart illustrating operation of the compression system.

### DESCRIPTION OF THE PREFERRED EMBODIMENTS

Hereinafter, one or more embodiments of the present invention will be described with reference to the drawings. However, the scope of the invention is not limited to the disclosed embodiments. Note that the same reference numerals will be used to represent the same configurations in each figure, and description thereof will be omitted as necessary. In the present specification, reference numerals without indices will be used to represent collective terms, and reference numerals with indices will be used to represent individual configurations.

A compression system in the embodiment includes an oil supply compressor and an oil mist measurement device configured to measure the concentration (an oil mist concentration) of oil mist in compressed gas output from the oil supply compressor. The oil mist measurement device includes a first detection section configured to detect the pressure of a measurement target which is the compressed gas, a second detection section configured to irradiate the measurement target with detection light to detect the intensity of light scattered by the oil mist in the measurement target, and a concentration processing section configured to correct the scattered light intensity detected by the second detection section based on the pressure detected by the first detection section to obtain a corrected scattered light intensity thereby obtaining the oil mist concentration based on the obtained corrected scattered light intensity. The compression system and the oil mist measurement device provided in the compression system will be more specifically described below.

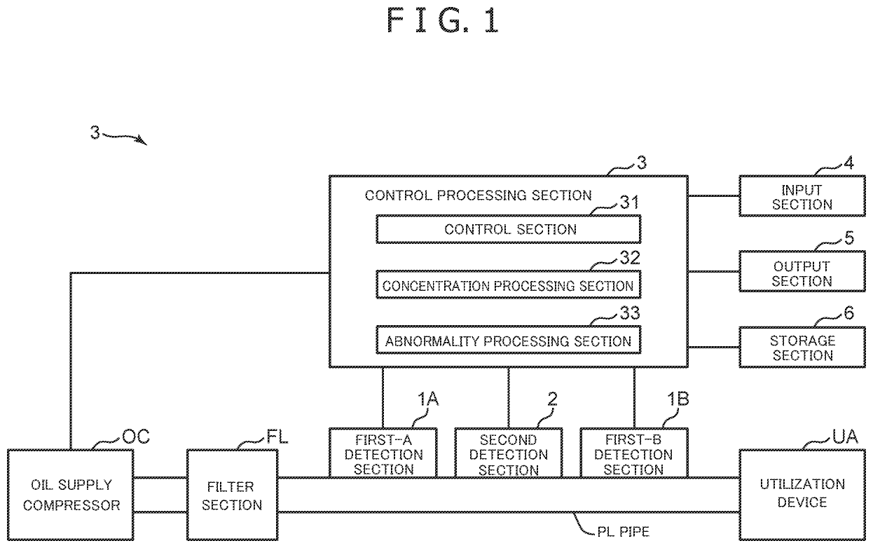

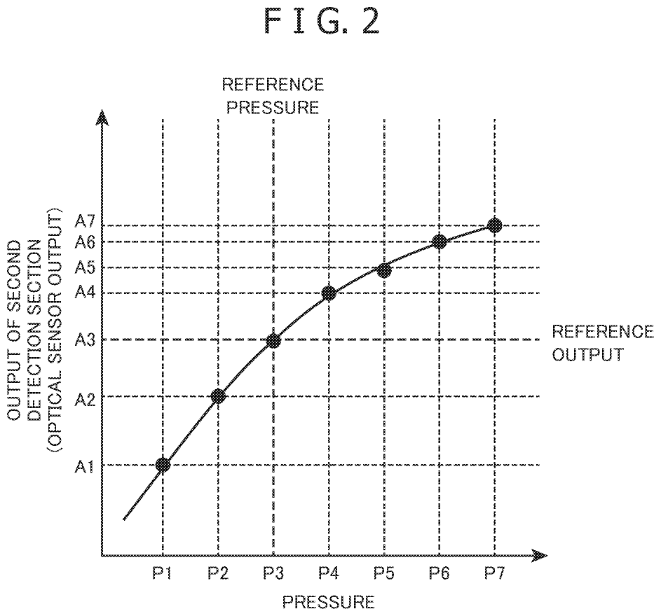

FIG. 1 is a block diagram illustrating the configuration of the compression system in the embodiment. FIG. 2 is a graph for describing a correction method in oil mist concentration calculation. The horizontal axis of FIG. 2 represents a measurement target (compressed gas) pressure, and the vertical axis of FIG. 2 represents the output of the second detection section (the output of an optical sensor).

A compression system S in the embodiment includes an oil supply compressor OC, a filter section FL, a first-A detection section 1A, a first-B detection section 1B, a second detection section 2, a control processing section 3, an input section 4, an output section 5, and a storage section 6.

The oil supply compressor OC is a device connected to the control processing section 3 and configured to supply, according to control by the control processing section 3, oil into gas to compress the gas for the purpose of lubrication, compression heat removal, etc. The oil supply compressor OC may be a compressor type for injecting oil into a compression chamber, and examples thereof include an oil-cooling gas compressor (e.g., an oil-cooling screw compressor and a reciprocating compressor). The gas compressed in the oil supply compressor OC flows in a pipe PL through the filter section FL, and is supplied to a predetermined device (a utilization device) UA utilizing the compressed gas.

The filter section FL is a device arranged, between the oil supply compressor OC and the utilization device UA, on the pipe PL in which the gas compressed in the oil supply compressor OC flows and configured to remove oil mist from the compressed gas such that an oil content falls below an acceptable oil content of the utilization device UA. The filter section FL includes, for example, a filter configured to collect the oil mist.

The first-A and first-B detection sections 1A, 1B are devices arranged in this order from an upstream side to a downstream side on the pipe PL, connected to the control processing section 3, and configured to detect, according to the control by the control processing section 3, the pressure of a measurement target which is the compressed gas flowing in the pipe PL. The first-A and first-B detection sections 1A, 1B each include pressure sensors. The first-A and first-B detection sections 1A, 1B output the pressures (pressure signals) as detection results to the control processing section 3.

The second detection section 2 is an optical device arranged between the first-A detection section 1A and the first-B detection section 1B on the pipe PL, connected to the control processing section 3, and configured to irradiate, according to the control by the control processing section 3, the measurement target which is the compressed gas flowing in the pipe PL with detection light to detect the intensity of light scattered by the oil mist in the measurement target. The second detection section 2 includes, for example, a light source such as a light-emitting diode and a light receiving element such as a photodiode, the light source and the light receiving element being arranged in parallel with a predetermined distance. The light source irradiates the measurement target with the detection light at a predetermined irradiation angle such as 45 degrees. The light receiving element receives the scattered light at a predetermined light receiving angle such as 45 degrees to photoelectrically convert the scattered light, thereby outputting a light intensity signal at a level corresponding to the intensity of the scattered light. According to preliminary experiment, it has been confirmed in advance that in the second detection section 2 as described above, the level of the light intensity signal and an oil mist amount are proportional. to each other. Considering the volume of an effective irradiation area where the oil mist can be detected by irradiation with the detection light, an oil mist weight flow rate per unit time is obtained from the oil mist amount. The second detection section 2 outputs the scattered light intensity (the light intensity signal) as a detection result of the second detection section 2 to the control processing section 3.

Note that the second detection section 2 has been described above as the parallel arrangement type that the light source and the light receiving element are arranged in parallel, but may be of an opposed type that the light source and the light receiving element are arranged to face each other such that the detection light emitted from the light source is received by the light receiving element. In the opposed type, the intensity of the detection light attenuated due to scattering in the oil mist is detected so that the intensity of the scattered light can be detected.

The input section 4 is equipment connected to the control processing section 3 and configured to input, to the compression system S (an oil mist measurement device), various commands such as a command for instructing the start of measurement and various types of data necessary for measuring the oil mist, such as the name of the measurement target. Examples of the input section 4 include multiple input switches to which predetermined functions are assigned, a keyboard, and a mouse. The output section 5 is equipment connected to the control processing section 3 and configured to output, according to the control by the control processing section 3, the command and data input from the input section 4, the detection result of each detection section 1A, 1B, 2, an oil mist concentration measured by the oil mist measurement device of the compression system S, etc. Examples of the output section 5 include display devices such as a CRT display, a liquid crystal display, and an organic EL display and a printing device such as a printer. As described later, an abnormality regarding the oil mist concentration is informed to the output section 5.

Note that the input section 4 and the output section 5 may form a so-called touch panel. In the case of forming this touch panel, the input section 4 is, for example, a resistive or capacitive position input device configured to detect and input an operation position, and the output section 5 is a display device. In this touch panel, the position input device is provided on a display surface of the display device, and one or more input content candidates which can be input to the display device are displayed. When a user touches a display position at which intended input contents are displayed, the position input device detects such a position, and the contents displayed at the detected position are, as user's operation input contents, input to the compression system S (the oil mist measurement device). The user can easily intuitively understand input operation on this touch panel, and therefore, the user-friendly compression system S (the oil mist measurement device) is provided.

The storage section 6 is a circuit connected to the control processing section 3 and configured to store various predetermined programs and various types of predetermined data according to the control by the control processing section 3. Examples of the various predetermined programs include a control program for controlling each section 1A, 1B, 2, 4 to 5, OC of the compression system S according to the function of each section, a concentration processing program for obtaining a corrected scattered light intensity by correcting the scattered light intensity detected by the second detection section 2 based on the pressures detected by the first-A and first-B detection sections 1A, 1B to obtain the oil mist concentration based on the obtained corrected scattered light intensity, and a control processing program such as an abnormality processing program for obtaining a cumulative oil mist amount accumulated over predetermined time based on the oil mist concentration obtained by the concentration processing program to perform predetermined abnormality processing in a case where the obtained cumulative oil mist amount exceeds a predetermined threshold (a determination threshold). The various types of predetermined data include data necessary for executing each program, such as the detection result of each detection section 1A, 1B, 2, the determination threshold, the volume of the effective irradiation area, predetermined first to third correspondences used for correction and oil mist concentration calculation. In the present embodiment, the first correspondence is a correspondence between a change in the measurement target pressure and a change in the output of the second detection section 2 for correction. The second correspondence is a correspondence between the level of the light intensity signal of the second detection section 2 and the oil mist amount. The third correspondence is a correspondence, in consideration of the volume of the effective irradiation area, between a compressed gas weight flow rate per unit time and the compressed gas pressure. Note that for the third correspondence in this example, the temperature of the compressed gas is a predetermined constant value. However, in a case where the compressed gas temperature fluctuates, the third correspondence may be a correspondence, in consideration of the volume of the effective irradiation area, among the compressed gas weight flow rate per unit time and the pressure and temperature of the compressed gas, and a temperature sensor configured to detect the compressed gas temperature may be further arranged on the pipe PL.

Examples of the storage section 6 as described above include a read. only memory (ROM) as a non-volatile storage element and an electrically erasable programmable read only memory (EEPROM) as a rewritable non-volatile storage element. Further, the examples of the storage section 6 include a random access memory (RAM) as a so-called working memory configured for the control processing section 3 to store, e.g., data generated. during execution of the predetermined program.

The control processing section 3 is a circuit configured to control each section 1A, 1B, 2, 4 to 5, OC of the compression system S according to the function of each section to obtain the oil mist concentration by correction of the scattered light intensity based on the pressure, thereby performing the predetermined abnormality processing based on the cumulative oil mist amount. The control processing section 3 includes, for example, a central processing unit (CPU) and a peripheral circuit thereof. The control processing program is executed such that a control section 31, a concentration processing section 32, and an abnormality processing section 33 are functionally implemented in the control processing section 3.

The control section 31 controls each section 1A, 1B, 2, 4 to 5, OC of the compression system S according to the function of each section, and is responsible for control across the entirety of the compression system S.

The concentration processing section 32 corrects the scattered light intensity (the light intensity signal) detected by the second detection section 2 based on the pressures (the pressure signals) detected by the first-A and first-B detection sections 1A, 1B to obtain the corrected scattered light intensity, thereby obtaining the oil mist concentration based on the obtained corrected scattered light intensity.

In the present embodiment, the concentration processing section 32 performs such correction by means of the first correspondence between the change in the measurement target (the compressed gas) pressure and the change in the output of the second detection section 2. One example of the first correspondence is illustrated in FIG. 2. In the example illustrated in FIG. 2, the output of the second detection section 2 non-linearly increases as the pressure of the compressed gas increases, and the increment thereof gradually decreases as the pressure of the compressed gas increases. Such a first correspondence is actually measured in advance, and as described above, is stored in advance in the storage section 6. In such a first correspondence, in a case where the output of the second detection section 2 at a predetermined reference pressure P3 set in advance is at a level (a reference level) A3, a scattered light intensity (a light intensity signal) Ad detected by the second detection section 2 is multiplied by a value obtained in such a manner that the reference level A3 is divided by a level Ak corresponding, in the first correspondence, to pressures Pk detected by the first-A and first-B detection sections 1A, 1B. In this manner, the scattered light intensity (the light intensity signal) Ad detected by the second detection section 2 is corrected, and a corrected scattered light intensity Ac is obtained. (Ac=(A3/Ak)×Ad). For example, in a case where the pressures detected by the first-A and first-B detection sections 1A, 1B are a pressure P2 (e.g., the average of the pressures detected by the first-A and first-B detection sections 1A, 1B is the pressure (an average pressure) P2), the concentration processing section 32 multiplies, by the scattered light intensity (the light intensity signal) Ad detected by the second detection section 2, a value A3/A2 obtained. in such a manner that the reference level A3 is divided by a level A2 corresponding to the pressure P2 in the first correspondence. In this manner, the concentration processing section 32 obtains the corrected scattered light intensity Ac(Ac=(A3/A2)×Ad).

Then, the concentration processing section 32 obtains the oil mist weight flow rate per unit time from the corrected scattered light intensity Ac by using the second correspondence and the volume of the effective irradiation area, and obtains the compressed gas weight flow rate per unit time from the pressures Pk detected by the first-A and first-B detection sections 1A, 1B by using the third correspondence. The concentration processing section 32 obtains the oil mist concentration from the obtained compressed gas weight flow rate and the obtained oil mist weight, flow rate per unit time ((the oil mist concentration [ppmwt])=(the oil mist weight flow rate per unit time)/(the compressed gas weight flow rate per unit time)×100, 1 ppm=0.0001%).

The abnormality processing section 33 obtains the cumulative oil mist amount accumulated over the predetermined time based on the oil mist concentration obtained by the concentration processing section 32, and performs the predetermined abnormality processing in a case where the obtained cumulative oil mist amount exceeds the determination threshold. More specifically, the concentration processing section 32 obtains the oil mist concentration at a predetermined sampling interval set in advance as described above, and the abnormality processing section 33 obtains the current cumulative oil mist amount by addition of the oil mist concentration currently obtained by the concentration processing section 32 to the previous cumulative oil mist amount. As a result of comparison between the obtained. current cumulative oil mist amount and the determination threshold, in a case where the current cumulative oil mist amount exceeds the determination threshold, the predetermined abnormality processing is performed for handling an oil mist abnormality. The predetermined time is, for example, the preset interval of maintenance of the filter section FL, and is reset when maintenance of the filter section FL is performed. The predetermined abnormality processing is, for example, the processing for reducing the rotational speed of the oil supply compressor CC. In this case, the abnormality processing section 33 notifies the control section 31 of the sensed abnormality, and the control section 31 controls the oil supply compressor OC to decrease the rotational speed thereof. Alternatively the predetermined abnormality processing is, for example, the processing for stopping the oil supply compressor OC. In this case, the abnormality processing section 33 notifies the control section 31 of the sensed abnormality and the control section 31 controls the oil supply compressor CC to stop operation thereof. Alternatively, the predetermined abnormality processing is, for example, the processing for notifying the abnormality regarding the oil mist concentration from the output section 5. In this case, the abnormality processing section 33 notifies the control section 31 of the sensed abnormality, and the control section 31 causes the output section 5 to output the abnormality regarding the oil mist concentration. For example, in a case where the output section 5 is the display device, a message indicating the abnormal oil mist concentration is displayed. Alternatively, in a case where the output section 5 is, for example, a lamp, the lamp is turned on.

in the compression system S with the configuration as described. above, the first-A and first-B detection sections 1A, 1B, the second detection section 2, and the concentration processing section 32 form the oil mist measurement device, and are one example of the oil mist measurement device.

The control processing section 3, the input section 4, the output section 5, and the storage section 6 can be implemented by, e.g., a desktop or laptop computer. The computer implementing these sections 3 to 6 may be, for example, arranged in an operation room of the oil supply compressor OC and be incorporated into a console of the oil supply compressor OC (also used as the console). Alternatively, the computer may be provided as a body separated from the console. In the case of the separated body, the control section 31 controls the oil supply compressor OC via the console thereof when the abnormality processing is executed.

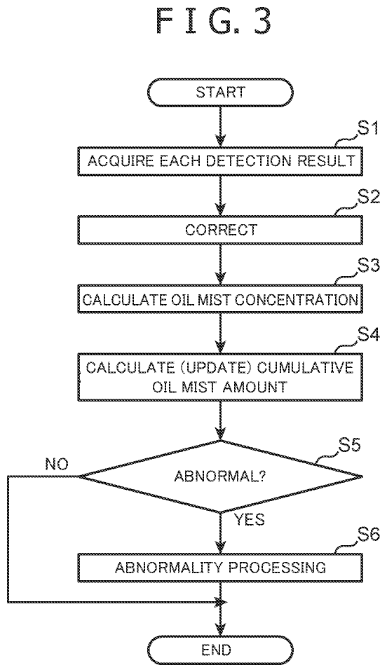

Next, operation of the present embodiment will be described. FIG. 3 is a flowchart illustrating operation of the compression system.

When power is supplied to the compression system S including the oil mist measurement device as configured above, the compression system S initializes each section as necessary and starts operating each section. By execution of the control processing program, the control section 31, the concentration processing section 32, and the abnormality processing section 33 are functionally implemented in the control processing section 3. The compression system S repeatedly executes each of the following types of processing at the predetermined sampling interval, thereby monitoring the oil mist concentration.

In FIG. 3, the compression system S first acquires the detection result from each detection section 1A, 1B, 2 by the control processing section 3 (S1).

Next, the compression system S corrects, by the concentration processing section 32 of the control processing section 3, the scattered light intensity (the light intensity signal) detected by the second detection section 2 (S2). More specifically, the concentration processing section 32 obtains, in the present embodiment, the average pressure Pk from the pressures detected b4 the first-A and first-B detection sections 1A, 1B, and obtains the level Ak corresponding to the obtained average pressure Pk in the first correspondence. The concentration processing section 32 multiplies the scattered light intensity (the light intensity signal) Ad detected by the second detection section 2 by the value A3/Ak obtained by division of the reference level A3 by the obtained level Ak. In this manner, the concentration processing section 32 obtains the corrected scattered light intensity Ac(Ac=(A3/Ak)×Ad).

Next, the compression system S calculates the oil mist concentration by the concentration processing section 32 (S3). More specifically, in the present embodiment, the concentration processing section 32 obtains the oil mist weight flow rate per unit time from the corrected scattered light intensity Ac obtained by the processing 52 by using the second. correspondence and the volume of the effective irradiation area, and obtains the compressed gas weight flow rate per unit time from the average pressure Pk obtained by the processing S2 by using the third correspondence. The concentration processing section 32 obtains the oil mist concentration from the obtained compressed gas weight flow rate and the obtained oil mist weight flow rate per unit time. Note that in the processing S3, the compression system S may be further configured such that the control processing section 3 causes the output section 5 to output the obtained oil mist concentration.

Next, the compression system S calculates, by the abnormality processing section 33 of the control processing section 3, the current cumulative oil mist amount (S4). More specifically in the present embodiment, the abnormality processing section 33 obtains the current cumulative oil mist amount by addition of the oil mist concentration obtained by the processing S3 to the previous cumulative oil mist amount.

Next, the compression system S determines the presence or absence of the abnormality by the abnormality processing section 33 (S5). More specifically, in the present embodiment, the abnormality processing section 33 compares the current cumulative oil mist amount obtained by the processing S4 and the determination threshold with each other, and determines the presence or absence of the abnormality by determining whether or not the current cumulative oil mist amount exceeds the determination threshold. As a result of such determination (as a result of comparison), in a case where the current cumulative oil mist amount exceeds the determination threshold, the abnormality processing section 33 determines that there is the abnormality (Yes). Next, the abnormality processing section 33 executes the predetermined abnormality processing (S6), and ends the present processing. On the other hand, as a result of determination, in a case where the current cumulative oil mist amount does not exceed the determination threshold, the abnormality processing section 33 determines that there is no abnormality (No), and ends the present processing.

As described above, in the oil mist measurement device of the compression system S and an oil mist measurement method performed by the oil mist measurement device, the intensity Ad of the light scattered by the oil mist in the compressed gas as the measurement target is corrected based on the measurement target pressure Pk, and in this manner, the oil mist concentration is obtained. Thus, the oil mist concentration can be obtained with higher accuracy.

In the oil mist measurement device and the oil mist measurement method, the output of the second detection section 2 at the predetermined reference pressure P3 set as a reference is obtained. Thus, using the pressures Pk detected by the first-A and first-B detection sections 1A, 1B and the first correspondence, the output Ad detected by the second detection section 2 can be converted into the output Ac at the reference pressure P3, and can be corrected.

According to the present embodiment, the compression system S including the oil mist measurement device can be provided. The compression system S performs the predetermined abnormality processing in a case where the cumulative oil mist amount exceeds the determination threshold, and therefore, can handle the oil mist abnormality.

Note that in the above-described embodiment, the compression system S (the oil mist measurement device) includes two first-A and first-B detection sections 1A, 1B, but may include any one of these sections.

In the above-described embodiment, the compression system S determines, by the abnormality processing section 33, whether or not the predetermined abnormality processing is to be performed based on the cumulative oil mist amount accumulated over the predetermined time. However, the abnormality processing section 33 may determine whether or not the predetermined abnormality processing is to be performed based on an instantaneous value of the oil mist concentration. In this case, the abnormality processing section 33 performs the predetermined abnormality processing in a case where the oil mist concentration obtained by the concentration processing section 32 exceeds a predetermined threshold (an instantaneous value determination threshold) set in advance. More specifically, the abnormality processing section 33 causes the concentration processing section 32 to obtain the oil mist concentration at predetermined timing set in advance, and compares the oil mist concentration and the instantaneous value determination threshold with each other. As a result of comparison, in a case where the oil mist concentration exceeds the instantaneous value determination threshold, the abnormality processing section 33 performs the predetermined abnormality processing to handle the oil mist abnormality.

For expressing the present invention, the present invention has been properly and adequately described above through explanation of the embodiment with reference to the drawings. However, those skilled in the art shall recognize that the above-described embodiment can be easily changed and/or modified. Thus, as long as change or modification made by those skilled in the art is not at a level departing from the scope of the claims, such change or modification is interpreted as being included in the scope of the claims.

What is claimed is:

1. An oil mist measurement device comprising:
a first detection section configured to detect a measurement target pressure;
a second detection section configured to irradiate a measurement target with detection light to detect an intensity of light scattered by oil mist in the measurement target; and
a concentration processing section configured to correct the scattered light intensity detected by the second detection section based on the pressure detected by the first detection section to obtain a corrected scattered light intensity, thereby obtaining an oil mist concentration based on the obtained corrected scattered light intensity.

2. The oil mist measurement device according to claim 1,
wherein the concentration processing section performs the correction by using a correspondence between a change in the measurement target pressure and a change in an output of the second detection section.

3. An oil mist measurement method comprising:
a first detection step of detecting a measurement target pressure;
a second detection step of irradiating a measurement target with detection light to detect an intensity of light scattered by oil list in the measurement target; and
a concentration processing step of correcting the scattered light intensity detected at the second detection step based on the pressure detected at the first detection step to obtain a corrected scattered light intensity, thereby obtaining an oil mist concentration lased on the obtained corrected scattered light intensity.

4. A compression system comprising:
an oil supply compressor; and
the oil mist measurement device according to claim 1,
wherein the measurement target is gas compressed in the oil supply compressor.

5. The compression system according to claim 4, further comprising:
an abnormality processing section configured to obtain a cumulative oil mist amount accumulated over predetermined time based on the oil mist concentration detected by the oil mist measurement device, thereby performing predetermined abnormality processing in a case where the obtained cumulative oil mist amount exceeds a predetermined threshold.
